(19) United States
(12) Patent Application Publication
(10) Pub. No.: US 2026/0221222 A1
(43) Pub. Date: Jul. 30, 2026

(54) GENERATING AUGMENTED BIOLOGICAL DATASETS VIA BATCH CORRECTION TECHNIQUES

(71) Applicant: Recursion Pharmaceuticals, Inc., Salt Lake City, UT (US)

(72) Inventors: Alisandra Kaye DENTON, Montreal (CA); Finagnon Marc-Rolland Emmanuel NOUTAHI, Montreal (CA); Ihab BENDIDI, Paris (FR); Kian Runnels KENYON-DEAN, Toronto (CA); Yassir EL MESBAHI, Montreal (CA)

(21) Appl. No.: 19/041,584

(22) Filed: Jan. 30, 2025

(51) Int. Cl. G16B 20/00; G16B 40/20

(52) U.S. Cl. CPC G16B 20/00; G16B 40/20

(57) ABSTRACT

The present disclosure relates to systems, non-transitory computer-readable media, and methods for generating biological data augmentations utilizing batch correction techniques to train machine learning models. In particular, in some embodiments, the disclosed systems access a biological dataset comprising biological data samples corresponding to cells exposed to cell perturbations. Additionally, in some embodiments, the disclosed systems generate, utilizing a first batch correction technique for a first sample of the biological data samples, a first batch corrected data sample. Moreover, in some implementations, the disclosed systems generate, utilizing a second batch correction technique for a second sample of the biological data samples, a second batch corrected data sample. Furthermore, in some embodiments, the disclosed systems generate, from the biological dataset, an augmented biological dataset for training a machine learning model by combining the biological data samples, the first batch corrected data sample, and the second batch corrected data sample.

### BACKGROUND

Recent years have seen developments in hardware and software platforms for training and utilizing machine learning models for generating predictions. For example, existing systems utilize large volumes of training data to teach machine learning models to generate intelligent predictions corresponding to complex biological interactions between genes, compounds, and/or proteins. Despite these recent developments, existing systems suffer from a number of technical deficiencies, particularly with regard to accuracy, efficiency, and operational flexibility in implementing machine learning technologies.

### BRIEF SUMMARY

Embodiments of the present disclosure provide benefits and/or solve one or more problems in the art with systems, non-transitory computer-readable media, and methods for enhancing unimodal biological embeddings with additional biological data via cross-modal knowledge distillation. To illustrate, in some embodiments, the disclosed systems distill knowledge from a first biological modality (e.g., phenomic representations) to a second biological modality (e.g., transcriptomic representations) to enhance the predictive power of the second modality while preserving its interpretability. Additionally, in some implementations, the disclosed systems provide benefits and/or solve one or more problems in the art with systems, non-transitory computer-readable media, and methods for augmenting biological data via batch correction techniques. To illustrate, in some embodiments, the disclosed systems apply batch alignment to data samples (e.g., embeddings) as data augmentations for training machine learning models to generate representations of pretrained unimodal models to boost the robustness and effectiveness of the knowledge distillation process.

The following description sets forth additional features and advantages of one or more embodiments of the disclosed methods, non-transitory computer-readable media, and systems. In some cases, such features and advantages are evident to a skilled artisan having the benefit of this disclosure, or may be learned by the practice of the disclosed embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview of a transcriptomic mapping system in accordance with one or more embodiments.

FIG. 2 illustrates a Venn diagram of biological relationships in a transcriptomic embedding space and a phenomic embedding space in accordance with one or more embodiments.

FIG. 3 illustrates the transcriptomic mapping system using knowledge distillation to map embeddings from a transcriptomic feature space to a phenomic feature space in accordance with one or more embodiments.

FIG. 4 illustrates the transcriptomic mapping system generating an augmented biological dataset utilizing batch correction techniques in accordance with one or more embodiments.

FIG. 5 illustrates the transcriptomic mapping system generating batch corrected data samples using a variety of batch correction techniques in accordance with one or more embodiments.

FIG. 6 illustrates the transcriptomic mapping system generating an augmented biological dataset to train a machine learning model in accordance with one or more embodiments.

FIG. 7 illustrates the transcriptomic mapping system generating batch corrected datasets for a teacher model and a student model to train a mapping model in accordance with one or more embodiments.

FIG. 8 illustrates a table of experimental results for the transcriptomic mapping system in comparison with existing systems in accordance with one or more embodiments.

FIG. 9 illustrates a diagram of an environment in which a transcriptomic mapping system operates in accordance with one or more embodiments.

FIG. 10 illustrates a flowchart of a series of acts for generating an enhanced biological embedding and training a mapping model based on the enhanced biological embedding in accordance with one or more embodiments.

FIG. 11 illustrates a flowchart of a series of acts for generating an augmented biological dataset in accordance with one or more embodiments.

FIG. 12 illustrates a block diagram of an example computing device for implementing one or more embodiments of the present disclosure.

### DETAILED DESCRIPTION

This disclosure describes one or more embodiments of a transcriptomic mapping system that utilizes knowledge distillation to improve machine learning representations of cellular responses to biological perturbations. To illustrate, in some embodiments, the transcriptomic mapping system utilizes a teacher-student framework to train a mapping model that generates enhanced embeddings of biological information relating to cell perturbations (e.g., chemical compound treatments, gene knockout sequences, etc.). For example, in some embodiments, the transcriptomic mapping system adapts multimodal alignment techniques for cross-modal knowledge distillation, such as distilling phenomic knowledge into transcriptomic representations. In addition, in some implementations, the transcriptomic mapping system applies a biologically inspired data augmentation approach using batch correction.

Understanding cellular responses to perturbations, and in particular understanding biological relationships across different types of perturbations, can be a complex part of pharmaceutical compound discovery. The transcriptomic mapping system provides a multimodal approach that offers a comprehensive view of these biological relationships to facilitate identifying drug targets. For example, the transcriptomic mapping system provides insight into complex cellular interactions by analyzing data from multiple biological modalities. For instance, the transcriptomic mapping system can analyze data from various-omics approaches, including transcriptomics (e.g., measuring gene expression levels), phenomics (e.g., observing image phenotypic traits), and proteomics (e.g., studying protein structures and functions). While each of these modalities capture unique aspects of cellular behavior that provide a partial view of a larger biological picture, the transcriptomic mapping system can combine these perspectives to reveal previously unseen biological connections, thereby accelerating drug discovery.

More particularly, in some implementations, the transcriptomic mapping system uses weakly paired data between biological modalities to train models to operate on a single modality during inference. For example, the transcriptomic mapping system utilizes cross-modal knowledge distillation to transfer information from one modality to another. To illustrate, in some embodiments, the transcriptomic mapping system trains a mapping model that distills knowledge from microscopy image representations (phenomics) into gene expression count representations (transcriptomics). By transferring phenotypic knowledge into transcriptomic representations, the transcriptomic mapping system enhances the transcriptomic representations with additional valuable information, thereby boosting their utility in applications such as drug discovery without the need for paired data during inference. This approach enhances the predictive power of machine learning models while preserving the interpretability of transcriptomics data samples, thus boosting their effectiveness for discovering biological relationships.

As described in additional detail below, in some embodiments, the transcriptomic mapping system provides a novel recipe for distilling knowledge from one biological modality to another using a perturbation dataset in which modalities are weakly paired at the level of perturbation and cell type. In particular, in some implementations, the transcriptomic mapping system provides cross-modal knowledge distillation from phenomics to transcriptomics.

Moreover, and as also described in additional detail below, in some embodiments, the transcriptomic mapping system utilizes batch alignment techniques to generate augmented datasets of biological representations (e.g., transcriptomic representations) to enhance the performance and robustness of knowledge distillation methods. For example, in some implementations, the transcriptomic mapping system uses the augmented datasets during training of the mapping model to enhance the robustness of the mapping model and improve the mapping model's generation of enhanced biological embeddings.

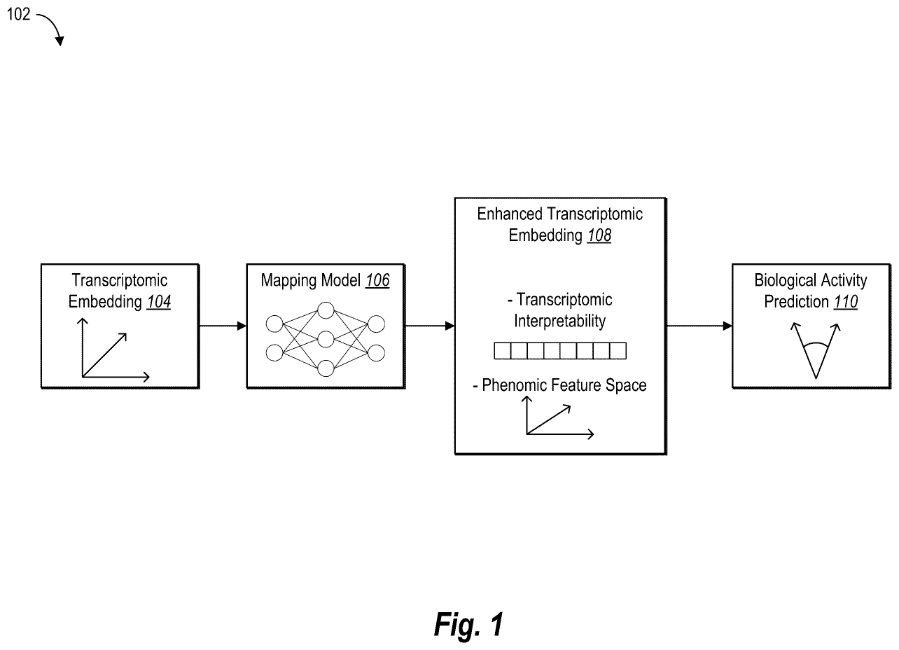

As just mentioned, in some embodiments, the transcriptomic mapping system utilizes a mapping model to map biological effects of cell perturbations from one embedding space to another. For instance, FIG. 1 illustrates a transcriptomic mapping system 102 utilizing a mapping model to generate an enhanced transcriptomic embedding from a transcriptomic embedding, and generating a biological activity prediction from the enhanced transcriptomic embedding, in accordance with one or more embodiments.

Specifically, FIG. 1 shows the transcriptomic mapping system 102 accessing a transcriptomic embedding 104 for a cell exposed to a cell perturbation. For example, in some cases, the transcriptomic mapping system 102 generates the transcriptomic embedding 104 from a transcriptomic profile for the cell perturbation (e.g., as described below in connection with the transcriptomic embedding 316). Alternatively, in some cases, the transcriptomic mapping system 102 obtains the transcriptomic embedding 104 from another system.

A cell perturbation includes a modification or treatment applied to a biological cell, such as by a chemical compound perturbation (e.g., a drug treatment) or a gene knockout perturbation. A perturbation can include a gene, small molecule (e.g., therapeutic compound or drug), biologic, or other treatment.

As further shown in FIG. 1, in some embodiments, the transcriptomic mapping system 102 uses a mapping model 106 to generate an enhanced transcriptomic embedding 108 from the transcriptomic embedding 104. For example, the transcriptomic mapping system 102 utilizes the mapping model 106 to convert the transcriptomic embedding 104 to a different embedding space. To illustrate, in some implementations, the transcriptomic embedding 104 is a numerical representation in a transcriptomic feature space. The transcriptomic mapping system 102 converts the transcriptomic embedding 104 to a phenomic feature space by generating the enhanced transcriptomic embedding 108 utilizing the mapping model 106. For instance, the enhanced transcriptomic embedding 108 is a numerical representation in the phenomic feature space.

In addition, in some implementations, the enhanced transcriptomic embedding 108 preserves transcriptomic interpretability (while also acquiring properties of phenomic embeddings). For instance, the transcriptomic mapping system 102 generates a feature vector that retains transcriptomic interpretability for generating a transcriptomic profile from the feature vector. For example, in some implementations, the transcriptomic mapping system 102 utilizes a transcriptomic decoder to reconstruct a transcriptomic profile from the enhanced transcriptomic embedding 108. Although the enhanced transcriptomic embedding 108 can be a feature vector in the phenomic feature space (and including phenomic biological information), the transcriptomic mapping system 102 can read the enhanced transcriptomic embedding 108 to extricate transcriptomic information that was initially found in the transcriptomic embedding 104. In this way, the transcriptomic mapping system 102 can distill additional biological information into a biological embedding that was not initially present in the biological embedding. Thus, during inference, the transcriptomic mapping system 102 enhances the biological information of a unimodal embedding with additional biological information from another modality.

A mapping model (or adapter) includes a machine learning model designed to generate enhanced biological embeddings from initial biological embeddings, imparting additional biological information that was initially not present in the initial embeddings. For example, in some embodiments, a mapping model is trained via knowledge distillation to impart characteristics to an embedding from a different dataset that was unseen during generation of the initial embedding.

A machine learning model includes a computer representation that is tunable (e.g., trained) based on inputs to approximate unknown functions used for generating corresponding outputs. In particular, in one or more embodiments, a machine learning model is a computer-implemented model that utilizes algorithms to learn from, and make predictions on, known data by analyzing the known data to learn to generate outputs that reflect patterns and attributes of the known data. For instance, in some cases, a machine learning model includes, but is not limited to, a neural network (e.g., a convolutional neural network, recurrent neural network, or other deep learning network), a decision tree (e.g., a gradient boosted decision tree), support vector learning, Bayesian networks, a transformer-based model, a diffusion model, or a combination thereof.

Similarly, a neural network includes a machine learning model that is trainable and/or tunable based on inputs to determine classifications and/or scores, or to approximate unknown functions. For example, in some cases, a neural network includes a model of interconnected artificial neurons (e.g., organized in layers) that communicate and learn to approximate complex functions and generate outputs based on inputs provided to the neural network. In some cases, a neural network refers to an algorithm (or set of algorithms) that implements deep learning techniques to model high-level abstractions in data. A neural network includes various layers such as an input layer, one or more hidden layers, and an output layer that each perform tasks for processing data. For example, a neural network includes a deep neural network, a convolutional neural network, a diffusion neural network, a recurrent neural network (e.g., an LSTM), a graph neural network, a transformer, or a generative adversarial neural network.

As additionally shown in FIG. 1, in some implementations, the transcriptomic mapping system 102 uses the enhanced transcriptomic embedding 108 to generate a biological activity prediction 110. For instance, the transcriptomic mapping system 102 determines a similarity prediction for the cell perturbation as compared to another perturbation based on a similarity score between the enhanced transcriptomic embedding 108 and another embedding corresponding to the other perturbation. For example, the transcriptomic mapping system 102 determines a similarity score between the enhanced transcriptomic embedding 108 and another enhanced transcriptomic embedding. The biological activity prediction 110 can then be used in a downstream task. For example, the transcriptomic mapping system 102 can use the biological activity prediction 110 to generate one or more additional predictions, or can coordinate with another system to initiate or advance one or more programs of drug discovery.

Moreover, in some embodiments, the transcriptomic mapping system 102 generates enhanced biological embeddings for various modalities, including other cross-modal interactions besides phenomics-to-transcriptomics knowledge distillation. For example, the transcriptomic mapping system 102 can generate enhanced proteomics embeddings via phenomics-to-proteomics knowledge distillation. As another example, the transcriptomic mapping system 102 can train a mapping model that generates enhanced phenomics embeddings from initial phenomics embeddings, whereby the transcriptomic mapping system 102 performs knowledge distillation from a first (e.g., more developed) phenomic machine learning model to a second (e.g., less developed) phenomic machine learning model. Additionally, the transcriptomic mapping system 102 can train a mapping model to generate enhanced embeddings that acquire additional biological information across acquisition modalities (e.g., knowledge distillation from cell painting image capture to brightfield image capture) or across cell types. Thus, while the description herein largely emphasizes phenomics-to-transcriptomics knowledge distillation, the transcriptomic mapping system 102 can perform knowledge distillation across other combinations of -omics or biological modalities.

As mentioned, conventional systems have a number of technical problems with regard to accuracy, operational flexibility, and efficiency of implementing computing devices. For example, existing systems are often inaccurate and inflexible in that they do not preserve information unique to each modality when aligning representations from different modalities. For instance, existing systems cannot preserve transcriptomic interpretability of biological embeddings while also integrating phenomic information into the embeddings. Moreover, existing systems often require consistently paired data across different modalities for producing accurate representations, yet such paired data is often unavailable and very challenging to collect. Furthermore, in the case of weakly paired biological data, natural variability between cells of the same perturbation can make it difficult to match features across samples, further resulting in inaccuracies of existing system outputs.

In addition, existing systems are inefficient because they often require excessive computational expense (e.g., expending excessive computing time, memory, processing operations, and network bandwidth) to align representations from different modalities. For example, existing systems often require processing of data from at least two modalities (e.g., both phenomics and transcriptomics) to align biological representations. Moreover, conventional systems often struggle with data sparsity problems. Indeed, conventional systems that train using biological data require excessive time and computational resources to generate training data. The prohibitive resources required to obtain training data often limits the accuracy and flexibility of conventional systems.

The transcriptomic mapping system 102 provides a variety of technical advantages relative to existing systems. For example, the transcriptomic mapping system 102 outperforms existing methods while preserving the interpretability of transcriptomic data. To illustrate, the transcriptomic mapping system 102 can provide enhanced accuracy of biosimilarity predictions by generating enhanced biological embeddings from unimodal encoders. For example, the transcriptomic mapping system 102 can combine rich phenotypic information from phenomics, such as morphological features, spatial organization, and cellular process indicators, with transcriptomics information, such as mitochondrial RNA gene counts, often associated with cell cycle measurements. In this way, the transcriptomic mapping system 102 can unlock a new layer of emergent biological synergy and insight, where the distillation from phenomics to transcriptomics yields biological information that neither modality could achieve alone, while still enabling inference on a single modality rather than requiring multimodal fusion.

Furthermore, the transcriptomic mapping system 102 improves upon existing systems with data augmentations via batch correction techniques. For instance, by generating batch correction augmentations, the transcriptomic mapping system 102 trains the mapping model to focus on biological information rather than batch effects. While the transcriptomic mapping system 102 can improve existing systems with these data augmentation techniques, the transcriptomic mapping system 102 provides even further enhancements over existing approaches by combining the batch correction data augmentations with the cross-modal knowledge distillation techniques described herein (e.g., phenomics-to-transcriptomics distillation). In this way, the transcriptomic mapping system 102 produces emergent synergies and capabilities that enrich biological representations with pathway-related insights not fully captured by existing systems.

Moreover, the transcriptomic mapping system 102 improves efficiency relative to conventional systems. Indeed, the transcriptomic mapping system 102 can reduce the amount of cross-modal data sampling to acquire biological information of cellular responses, thereby enhancing efficiency of computational biological investigations, such as computational drug discovery pipelines. Furthermore, in some embodiments, the transcriptomic mapping system 102 maps from one modality to another utilizing self-supervised distillation, which is more effective than using biological labels alone (because labels often lack comprehensive biological detail). Thus, by utilizing knowledge distillation to prepare a mapping model, the transcriptomic mapping system 102 can operate on unimodal embeddings at inference time, thereby avoiding the computational expense that would otherwise be required to process a second modality's data samples. For example, by converting transcriptomic embeddings to a phenomic feature space, the transcriptomic mapping system 102 provides enhanced efficiency over existing systems by distilling phenomic information into a transcriptomic embedding without needing to run and process phenomic experiments.

The transcriptomic mapping system 102 also improves efficiency by utilizing batch correction techniques to augment biological data sets. Indeed, the transcriptomic mapping system 102 can utilize multiple different batch correction techniques to generate significantly different data samples from an initial training set. This approach allows the transcriptomic mapping system 102 to multiply existing datasets multiple times over, significantly improving the available data for training machine learning models with significantly reduced time and computational expense.

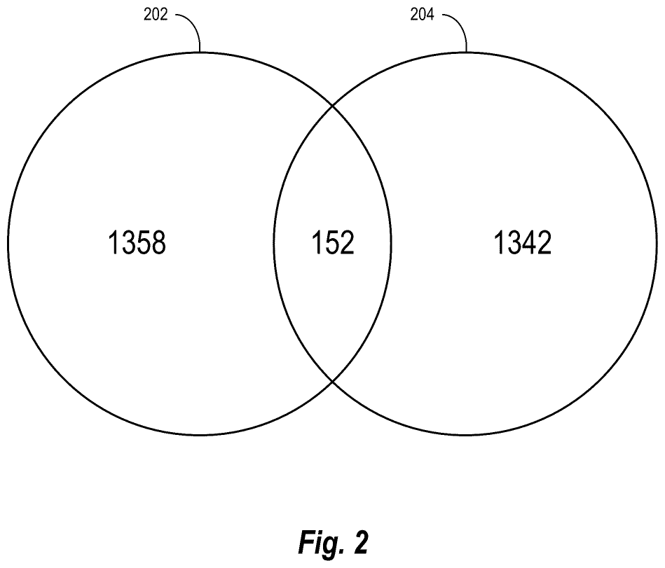

As mentioned, in some embodiments, the transcriptomic mapping system 102 transfers information from one biological embedding space to another. Moreover, in some cases, various biological embedding spaces capture different biological relationships and therefore have limited overlap (e.g., weak pairing). For instance, FIG. 2 illustrates a Venn diagram of biological relationships in a transcriptomic embedding space and a phenomic embedding space, in accordance with one or more embodiments.

Specifically, FIG. 2 shows a Venn diagram illustrating a comparison of a transcriptomic latent space 202 with a phenomic latent space 204. The transcriptomic latent space 202 and the phenomic latent space 204 represent, a particular threshold, relationships uncovered through analysis of the latent spaces of unimodal encoders of a transcriptomic machine learning model and a phenomic machine learning model, respectively. As demonstrated, the transcriptomic latent space captures a set of relationships that is largely distinct from the relationships captured by the phenomic latent space. In particular, FIG. 2 shows that, of 1358 transcriptomic relationships and of 1342 phenomic relationships retrieved with a threshold of the top one percent and bottom one percent for latent spaces of the unimodal encoders, just 152 are overlapping in the insights that each embedding modality uncovers.

By distilling knowledge from one modality (e.g., phenomics) to another modality (e.g., transcriptomics) that has little overlap, the transcriptomic mapping system 102 enhances discovery biology by imparting information from the one modality to embeddings of the other modality. For example, although phenomics and transcriptomics have limited overlapping biological information, the transcriptomic mapping system 102 utilizes knowledge distillation from phenomics to transcriptomics to provide embeddings from transcriptomics with additional, phenomic information.

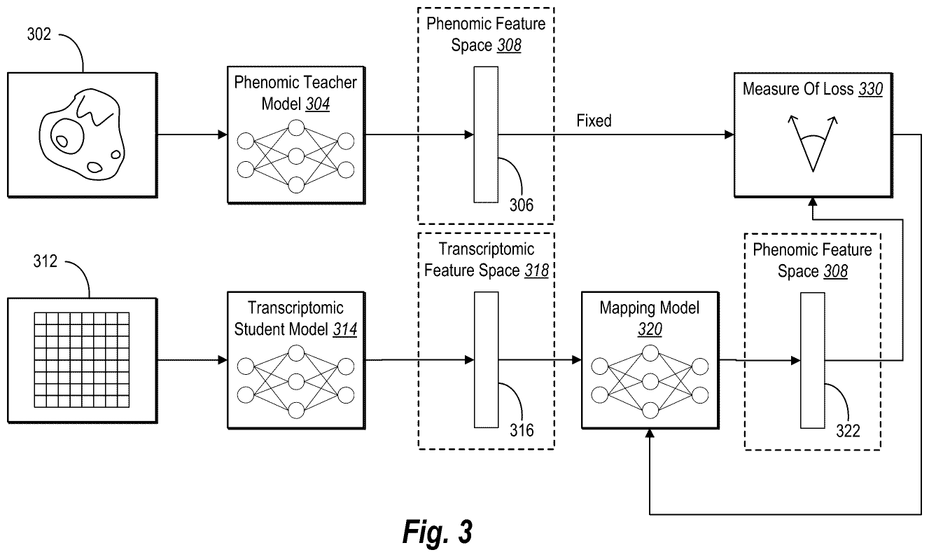

As discussed above, in some embodiments, the transcriptomic mapping system 102 trains a mapping model to map biological embeddings across feature spaces. For instance, FIG. 3 illustrates the transcriptomic mapping system 102 using knowledge distillation to map embeddings from a transcriptomic feature space to a phenomic feature space in accordance with one or more embodiments.

Specifically, FIG. 3 shows the transcriptomic mapping system 102 accessing a phenomic data sample 302 and a transcriptomic data sample 312. For example, the phenomic data sample 302 is a digital image portraying a first cell exposed to a cell perturbation. Similarly, the transcriptomic data sample 312 is a transcriptomic profile for a second cell exposed to a cell perturbation (e.g., the same cell perturbation as for the phenomic data sample 302).

In some cases, the transcriptomic mapping system 102 generates the phenomic data sample 302 by capturing the digital image of the first cell upon exposure to the cell perturbation in a laboratory environment. For example, the transcriptomic mapping system 102 coordinates with experimental device(s) to run experiments on cell samples by exposing the cell samples to one or more perturbations and capturing digital images of the cells to harness phenotypic information relating to the cell perturbations.

Additionally, in some cases, the transcriptomic mapping system 102 generates the transcriptomic data sample 312 by generating the transcriptomic profile for the second cell upon exposure to the cell perturbation in a laboratory environment. For example, the transcriptomic mapping system 102 coordinates with experimental device(s) to run experiments on cell samples by exposing the cell samples to one or more perturbations, detecting RNA transcription expression counts within the cells, and generating a data structure of the transcription expression counts to harness transcriptomic information relating to the cell perturbations.

In some implementations, the transcriptomic mapping system 102 uses a phenomic teacher machine learning model 304 to generate a phenomic embedding 306 from the phenomic data sample 302. For instance, the transcriptomic mapping system 102 processes the phenomic data sample 302 through an encoder of the phenomic teacher machine learning model 304 to generate a feature vector in a phenomic feature space 308. For example, in some implementations, the transcriptomic mapping system 102 generates the phenomic embedding 306 by utilizing a machine learning model trained to generate predicted cell representations from masked cell representations as described in U.S. Pat. No. 12,119,090, titled UTILIZING MASKED AUTOENCODER GENERATIVE MODELS TO EXTRACT MICROSCOPY REPRESENTATION AUTOENCODER EMBEDDINGS, issued on Oct. 15, 2024 (hereinafter the '090 patent), which is incorporated by reference herein in its entirety. Moreover, in some embodiments, the transcriptomic mapping system 102 generates the phenomic embedding 306 utilizing techniques described in U.S. patent application Ser. No. 18/526,707, titled UTILIZING MACHINE LEARNING MODELS TO SYNTHESIZE PERTURBATION DATA TO GENERATE PERTURBATION HEATMAP GRAPHICAL USER INTERFACES, filed on Dec. 1, 2023 (hereinafter the '707 application), which is incorporated by reference herein in its entirety.

Relatedly, in some implementations, the transcriptomic mapping system 102 uses a transcriptomic student machine learning model 314 to generate a transcriptomic embedding 316 from the transcriptomic data sample 312. For instance, the transcriptomic mapping system 102 processes the transcriptomic data sample 312 through an encoder of the transcriptomic student machine learning model 314 to generate a feature vector in a transcriptomic feature space 318. The transcriptomic mapping system 102 can utilize a variety of different machine learning models or architectures for the transcriptomic student model 314, including, but not limited to, sc VI, Geneformer, scGPT, CellPLM, Universal Cell Embeddings or “UCE,” scBERT, and/or scVAEIT. The transcriptomic mapping system 102 can also utilize the model described in the '090 patent to generate a transcriptomic embedding from a transcriptomic profile.

Moreover, as shown in FIG. 3, the transcriptomic mapping system 102 uses a mapping model 320 (e.g., the mapping model 106) to generate an enhanced transcriptomic embedding 322 from the transcriptomic embedding 316. For instance, the transcriptomic mapping system 102 processes the transcriptomic embedding 316 through the mapping model 320 to generate a feature vector in the phenomic feature space 308 that preserves transcriptomic interpretability of the transcriptomic embedding 316.

Additionally, as shown in FIG. 3, in some implementations, the transcriptomic mapping system 102 compares the enhanced transcriptomic embedding 322 to the phenomic embedding 306. For example, the transcriptomic mapping system 102 determines a measure of loss 330 (e.g., a cosine similarity or other feature space distance metric) between the enhanced transcriptomic embedding 322 and the phenomic embedding 306.

Moreover, in some embodiments, the transcriptomic mapping system 102 uses the comparison (i.e., the measure of loss 330) of the enhanced transcriptomic embedding 322 to the phenomic embedding 306 to train the mapping model 320. For example, the transcriptomic mapping system 102 adjusts parameters of the mapping model 320 to improve the measure of loss 330 (e.g., on a subsequent training iteration).

Furthermore, in some embodiments, the transcriptomic mapping system 102 repeats this training process numerous times to tune the mapping model 320. For example, the transcriptomic mapping system 102 compares a second enhanced transcriptomic embedding for a second cell perturbation to a second phenomic embedding for the second cell perturbation to determine an updated (or new) measure of loss. The transcriptomic mapping system 102 then further adjusts the parameters of the mapping model 320 to improve the measure of loss.

Moreover, in some implementations, the transcriptomic mapping system 102 tunes the mapping model 320 with fixed teacher embeddings. For example, the transcriptomic mapping system 102 fixes the phenomic embeddings 306 generated by the phenomic teacher machine learning model 304. In this way, the transcriptomic mapping system 102 adjusts the parameters of the mapping model 320 to learn to map the transcriptomic embeddings 316 to the phenomic feature space 308.

To further illustrate, in some embodiments, the transcriptomic mapping system 102 considers two biological data modalities: a teacher modality T and a student modality S, each offering distinct perspectives on cellular behavior. Let [symbol] and [symbol] represent the datasets from these modalities, respectively. The samples

$${x}_{T}^{( i )} ∈ {𝒳}_{T} and {x}_{S}^{( i )} ∈ {𝒳}_{S}$$

correspond to the same biological perturbation and cell line but are not perfectly aligned due to sample differences. Each sample is annotated with weak labels p (perturbation) and/(cell line). Both datasets are organized into biological batches [symbol] ∈{b_{T,1}, b_{T,2}, . . . , [symbol] } and [symbol] ∈{b_{S,1}, b_{S,2}, . . . , [symbol] }. Each batch b_{T,k}∈ [symbol] and b_{S,m}∈ [symbol] consists of a set of samples

$${\{ {x}_{T , k}^{( j )} \}}_{j = 1}^{{N}_{T , k}} and {\{ {x}_{S , m}^{( j )} \}}_{j = 1}^{{N}_{S , m}} ,$$

where N_{T,k} and N_{S,m} denote the number of samples in batch b_{T,k} and b_{S,m}, respectively, and ∃i such that N_{M,k}≠N_{M′,i}, ∀(M, M′)∈T, S. Each batch includes, in addition to perturbed samples, a number of control (unperturbed) samples, denoted by

$${\{ {x}_{T , k}^{( c )} \}}_{c = 1}^{{C}_{T , k}} and {\{ {x}_{S , m}^{( c )} \}}_{c = 1}^{{C}_{S , m}}$$

for C_{T,k}≥2 and C_{S,m}≥2.

Given the limited availability of paired data, in some implementations, the transcriptomic mapping system 102 utilizes pretrained and frozen unimodal encoders E_{T}: [symbol] → [symbol] and E_{S}: [symbol] → [symbol] . These encoders produce embeddings

$${z}_{T}^{( i )} = {E}_{T} ( {x}_{T}^{( i )} ) and {z}_{S}^{( i )} = {E}_{S} ( {x}_{S}^{( i )} )$$

for the teacher and student modalities, respectively. In some embodiments, the transcriptomic mapping system 102 learns a mapping function ƒ_{S}: [symbol] → [symbol] that maps the student embeddings into the teacher embedding space, resulting in adapted embeddings

$${h}_{S}^{( i )} = fs ( {z}_{S}^{( i )} )$$

that incorporate information from the teacher modality T.

Furthermore, in some embodiments, the transcriptomic mapping system 102 uses pretrained and frozen unimodal encoders (e.g., the phenomic teacher model 304 and the transcriptomic student model 314) to generate the embeddings z_{T} and z_{S}. The output of the teacher encoder is fixed, and an adapter function ƒ_{S} (e.g., the mapping model 320) is trained on the student modality to produce transformed embeddings h_{S}, aligning them with the teacher embeddings z_{T}.

In some implementations, the transcriptomic mapping system 102 utilizes a loss function (e.g., to determine the measure of loss 330) defined as

$$ℒ = - \frac{1}{N} {\sum }_{i = 1}^{N} [ log \frac{exp ( sim ( {h}_{S}^{( i )} , {z}_{T}^{( i )} ) / τ )}{{\sum }_{j = 1}^{N} exp ( sim ( {h}_{S}^{( i )} , {z}_{T}^{( j )} ) / τ )} ]$$

$$where$$

$$sim ( u , v ) = \frac{{u}^{⊤} v}{\vert u \vert \vert v \vert}$$

represents cosine similarity and τ>0 is a temperature hyperparameter.

Thus, in some embodiments, the transcriptomic mapping system 102 performs one-way knowledge transfer from the teacher to the student without altering the teacher's representations, thereby mitigating mutual drift and information feedback from the student to the teacher, and reducing dependence on the weak labels.

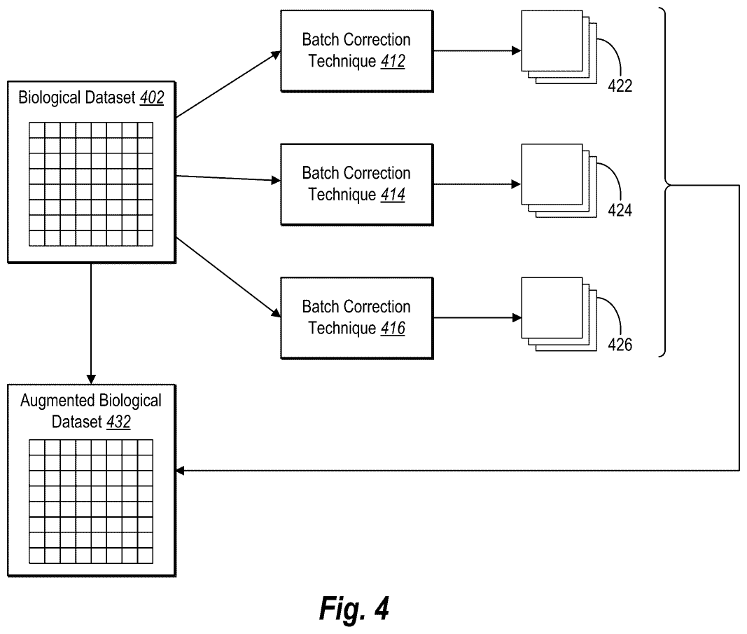

As mentioned above, in some embodiments, the transcriptomic mapping system 102 applies batch correction techniques as data augmentations for biological data. For instance, FIG. 4 illustrates the transcriptomic mapping system 102 generating an augmented biological dataset utilizing batch correction techniques in accordance with one or more embodiments.

Specifically, FIG. 4 shows the transcriptomic mapping system 102 accessing a biological dataset 402 comprising biological data samples corresponding to cells exposed to cell perturbations. For instance, the biological dataset 402 includes transcriptomic profiles for the cells exposed to the cell perturbations. In some cases, the biological dataset 402 includes other modes of biological data, such as phenomic data, invivomic data, and/or other -omic data.

Additionally, FIG. 4 shows the transcriptomic mapping system 102 applying batch correction techniques to the biological dataset 402. For example, the transcriptomic mapping system 102 utilizes a first batch correction technique 412 for a first sample of the biological data samples in the biological dataset 402 to generate a first batch corrected data sample 422. Likewise, in some embodiments, the transcriptomic mapping system 102 utilizes a second batch correction technique 414 for a second sample of the biological data samples to generate a second batch corrected data sample 424. Similarly, in some implementations, the transcriptomic mapping system 102 utilizes a third batch correction technique 416 for a third sample of the biological data samples to generate a third batch corrected data sample 426.

Moreover, as shown in FIG. 4, in some embodiments, the transcriptomic mapping system 102 generates an augmented biological dataset 432. For instance, the transcriptomic mapping system 102 combines the biological data samples of the biological dataset 402 with the batch corrected data samples. More particularly, in some embodiments, the transcriptomic mapping system 102 combines the biological data samples, the first batch corrected data sample 422, the second batch corrected data sample 424, and the third batch corrected data sample 426 to generate the augmented biological dataset 432 from the biological dataset 402.

Furthermore, in some implementations, the transcriptomic mapping system 102 generates the augmented biological dataset 432 for training a machine learning model. For example, the transcriptomic mapping system 102 generates the augmented biological dataset 432 for training the mapping model 320 described above in connection with FIG. 3.

To further illustrate, in some embodiments, the transcriptomic mapping system 102 mitigates batch effects due to variations in experimental conditions that can introduce unwanted variability and obscure true biological signals. For example, the transcriptomic mapping system 102 incorporates various batch correction methods as data augmentation techniques applied directly to the student modality representations. In particular, the transcriptomic mapping system 102 utilizes a function A: [symbol] → [symbol] that randomly applies a batch correction transformation from a set [symbol] to the student embeddings

$${z}_{S}^{( i )} .$$

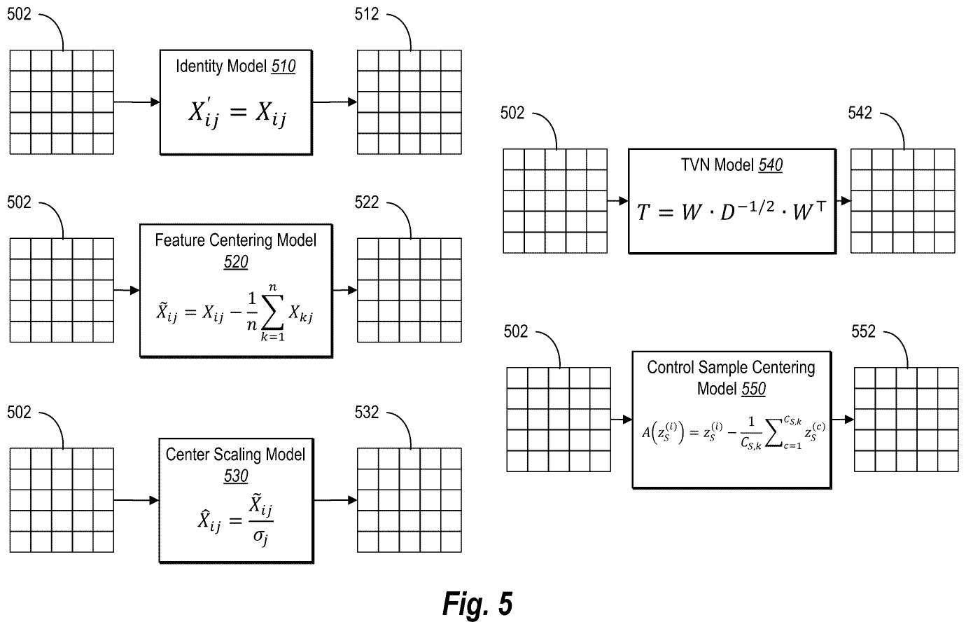

Examples of batch correction transformations within the set [symbol] are described below in connection with FIG. 5. In some implementations, the augmented embeddings (e.g., the augmented biological dataset 432), represented as

$${z}_{S , A}^{( i )} = A ( {z}_{S}^{( i )} ) ,$$

are input to the adapter f_{S} for cross-modal knowledge distillation.

As mentioned, in some embodiments, the transcriptomic mapping system 102 applies batch correction techniques to biological data samples. For instance, FIG. 5 illustrates the transcriptomic mapping system 102 generating batch corrected data samples using a variety of batch correction techniques in accordance with one or more embodiments.

Specifically, FIG. 5 shows the transcriptomic mapping system 102 accessing a biological dataset 502 comprising biological data samples corresponding to cells exposed to cell perturbations. Additionally, FIG. 5 shows the transcriptomic mapping system 102 applying batch correction techniques to the data samples of the biological dataset 502 to generate batch corrected data samples for augmented biological datasets. To illustrate, in the following descriptions, the transcriptomic mapping system 102 applies batch correction techniques to data samples denoted as Xᵢⱼ within a feature matrix X∈ [symbol] , where n is the number of samples and m is the number of features.

For example, the transcriptomic mapping system 102 utilizes an identity model 510 to generate batch corrected data samples 512. The identity model 510 yields an output that matches the input. To illustrate, for a data sample Xᵢⱼ, the identity model 510 generates a batch corrected data sample 512 of

$${X}_{ij}^{′} = {X}_{ij} .$$

As another example, the transcriptomic mapping system 102 utilizes a feature centering model 520 to generate batch corrected data samples 522. The feature centering model 520 adjusts the dataset such that each feature has a mean of zero. To illustrate, the feature centering model 520 subtracts the mean of each feature from the data. Thus, the feature centering model 520 generates a batch corrected data sample 522 of:

$${\hat{X}}_{ij} = {X}_{ij} - \frac{1}{n} {\sum }_{k = 1}^{n} {X}_{kj} ,$$

$$∀ i = 1 , … , n ,$$

$$∀ j = 1 , … , m$$

This step shifts the data so that each feature's mean is zero. In some embodiments, the transcriptomic mapping system 102 applies centering to a biological dataset to remove the influence of negative control embeddings, thereby facilitating the focus on perturbation effects.

For another example, the transcriptomic mapping system 102 utilizes a center scaling model 530 to generate batch corrected data samples 532. The center scaling model 530 is an extension of the feature centering model 520. The center scaling model 530 adjusts each feature so that it has unit variance. To illustrate, the center scaling model 530 scales a centered matrix \tilde{X} to generate a scaled matrix \hat{X} with batch corrected data samples 532 defined as

$${\hat{X}}_{ij} = \frac{{\hat{X}}_{ij}}{{σ}_{j}}$$

where

$${σ}_{j} = \sqrt{\frac{1}{n} {\sum }_{k = 1}^{n} {\tilde{X}}_{kj}^{2}}$$

represents the standard deviation of the jth feature. In some embodiments, the transcriptomic mapping system 102 uses center scaling to enhance comparability across features and support techniques that are influenced by the scale of data, such as principal component analysis (PCA).

As yet another example, the transcriptomic mapping system 102 utilizes a typical variation normalization (TVN) model 540 to generate batch corrected data samples 542. In some embodiments, the transcriptomic mapping system 102 uses typical variation normalization to enhance the representation of biological data by minimizing batch effects and accentuating subtle phenotypic differences. TVN is particularly relevant in high-content imaging screens and other scenarios with significant batch variability. The TVN model 540 computes the principal components of control samples (negative control conditions) to identify the primary directions of variation. The TVN model 540 performs principal component analysis on the centered control data \tilde{X}_{control} to obtain principal components {v₁, . . . , vₘ}, with each component representing a variance direction in the data space.

To illustrate, the TVN model 540 centers and scales the control data as follows:

$${\hat{X}}_{control} = \frac{{\hat{X}}_{control} - {μ}_{control}}{{σ}_{control}}$$

where μ_{control} and σ_{control} are the mean and standard deviation of the control embeddings. The TVN model 540 conducts PCA on \hat{X}_{control} to derive principal components. The TVN model 540 generates a matrix W∈ [symbol] that consists of columns that are the component vectors vⱼ. The TVN model 540 generates a transformation matrix T to normalize variance along each principal component axis:

$$T = W \cdot {D}^{- 1 / 2} \cdot {W}^{⊤}$$

where D is a diagonal matrix of the eigenvalues associated with the principal components. The TVN model 540 applies the transformation to all embeddings Xₐₗₗ as:

$${X}_{TVN} = T \cdot {X}_{all}$$

This reduces unwanted variation while emphasizing important biological differences, enabling a focus on subtle or rare phenotypic features without batch-related artifacts.

For still another example, the transcriptomic mapping system 102 utilizes a control sample centering model 550 to generate batch corrected data samples 552. The control sample centering model 550 centers the data samples around a control sample to mitigate batch effects. To illustrate, the control sample centering model 550 generates a batch corrected data sample 552 for the student embeddings, defined as:

$$A ( {z}_{S}^{( i )} ) = {z}_{S}^{( i )} - \frac{1}{{C}_{S , k}} {\sum }_{c = 1}^{{C}_{S , k}} {z}_{S}^{( c )}$$

where C_{S,k} are the control samples in batch b_{s,k}.

In some embodiments, the transcriptomic mapping system 102 selects one or more batch correction techniques from the options just described to generate batch corrected data samples. For example, the transcriptomic mapping system 102 utilizes the identity model 510, the feature centering model 520, or the center scaling model 530 to generate a first batch corrected data sample. Relatedly, in some examples, the transcriptomic mapping system 102 utilizes the TVN model 540 or the control sample centering model 550 to generate a second batch corrected data sample. The transcriptomic mapping system 102 can utilize a variety of different combinations of different batch correction techniques to generate an augmented data set.

Moreover, in some implementations, the transcriptomic mapping system 102 randomly selects one of the above-described batch correction techniques for generating augmented data for training a machine learning model, such as the mapping model 320 (as described in additional detail below), and selects the TVN model 540 to generate batch-corrected embeddings (e.g., batch corrected enhanced transcriptomic embeddings) at inference.

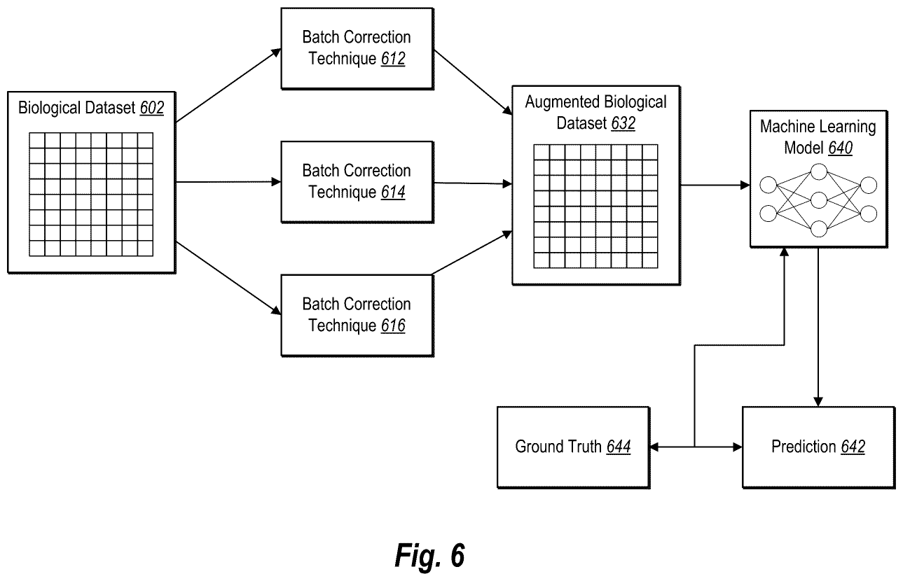

As mentioned, in some embodiments, the transcriptomic mapping system 102 trains a machine learning model utilizing augmented biological data. For instance, FIG. 6 illustrates the transcriptomic mapping system 102 generating an augmented biological dataset to train a machine learning model in accordance with one or more embodiments.

Specifically, FIG. 6 shows the transcriptomic mapping system 102 accessing a biological dataset 602 comprising biological data samples corresponding to cells exposed to cell perturbations. Moreover, FIG. 6 shows the transcriptomic mapping system 102 utilizing batch correction techniques 612, 614, and 616 to generate an augmented biological dataset 632 (e.g., using the techniques described above in connection with FIGS. 4 and 5).

Furthermore, FIG. 6 shows the transcriptomic mapping system 102 using the augmented biological dataset 632 to train a machine learning model 640. To illustrate, the transcriptomic mapping system 102 generates a biological prediction 642 for a cell perturbation from a first batch corrected embedding of the augmented biological dataset 632. Additionally, the transcriptomic mapping system 102 compares the biological prediction 642 to a ground truth 644 (e.g., to determine a measure of loss). Based on the comparison of the biological prediction 642 to the ground truth 644, the transcriptomic mapping system 102 adjusts parameters of the machine learning model 640 (e.g., to reduce the measure of loss on a subsequent training iteration).

Moreover, in some embodiments, the machine learning model 640 is an adapter, such as a mapping model for converting transcriptomic embeddings to a phenomic embedding space (e.g., such as the mapping model 320 described above). To illustrate, the transcriptomic mapping system 102 adjusts parameters of the mapping model to reduce the measure of loss.

As mentioned above, in some embodiments, the transcriptomic mapping system 102 generates biological data samples (e.g., for the biological dataset 602) by generating embeddings for cells exposed to cell perturbations. For example, the transcriptomic mapping system 102 utilizes an embedding model, such as a phenomic embedding machine learning model or a transcriptomic embedding machine learning model, to generate the embeddings. Moreover, the transcriptomic mapping system 102 generates batch corrected data samples for one or more of the data samples in the biological dataset 602 by generating batch corrected embeddings (e.g., using one or more batch correction techniques described herein) from the generated embeddings.

As mentioned, in some embodiments, the transcriptomic mapping system 102 extends the data augmentation techniques described above (e.g., in connection with FIGS. 4 and 5) to the knowledge distillation techniques described above (e.g., in connection with FIG. 3).

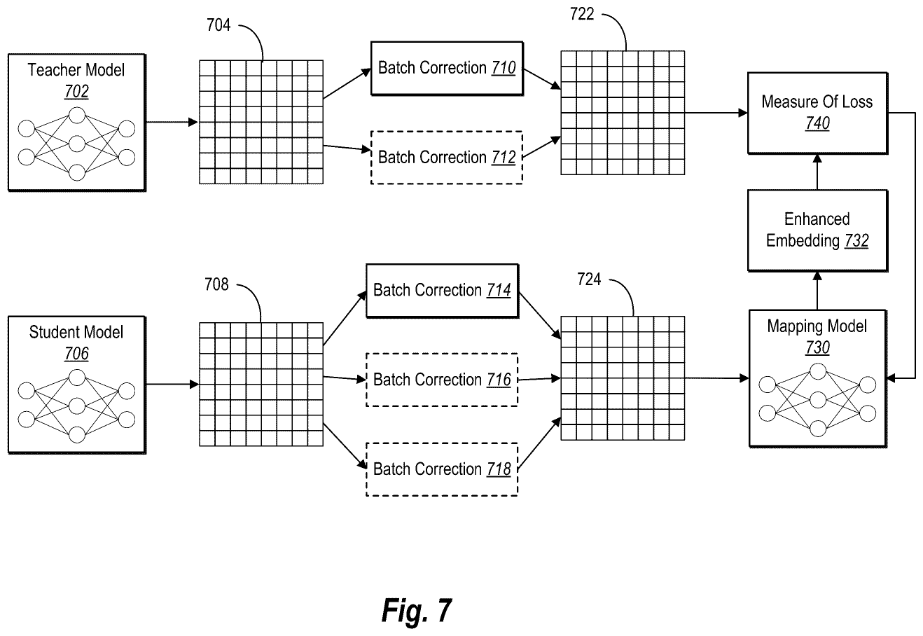

To illustrate, in some embodiments, the transcriptomic mapping system 102 tunes a machine learning model using knowledge distillation with augmented biological data. For instance, FIG. 7 illustrates the transcriptomic mapping system 102 generating batch corrected datasets for a teacher model and a student model to train a mapping model in accordance with one or more embodiments.

Specifically, FIG. 7 shows the transcriptomic mapping system 102 utilizing a teacher model 702 (e.g., the phenomic teacher model 304) to generate a teacher dataset 704. For example, the transcriptomic mapping system 102 generates, for the teacher dataset 704, phenomic embeddings from digital images portraying a first set of cells exposed to cell perturbations.

Additionally, FIG. 7 shows the transcriptomic mapping system 102 utilizing a student model 706 (e.g., the transcriptomic student model 314) to generate a student dataset 708. For instance, the transcriptomic mapping system 102 generates, for the student dataset 708, transcriptomic embeddings from transcriptomic profiles for a second set of cells exposed to the cell perturbations.

Moreover, FIG. 7 shows the transcriptomic mapping system 102 utilizing batch correction to generate augmented biological datasets from the teacher dataset 704 and the student dataset 708. For example, the transcriptomic mapping system 102 uses a first batch correction technique 710 to generate one or more first batch corrected data samples from the teacher dataset 704. In some embodiments, the transcriptomic mapping system 102 additionally uses a second batch correction technique 712 to generate one or more second batch corrected data samples from the teacher dataset 704. As described above in connection with FIG. 5, the transcriptomic mapping system 102 can select from a variety of batch correction techniques for this data augmentation process. The transcriptomic mapping system 102 adds these batch corrected data samples to an augmented teacher biological dataset 722.

To illustrate, the transcriptomic mapping system 102 generates a first batch corrected data sample by utilizing a typical variation normalization model (e.g., the TVN model 540) to adjust a teacher sample from the teacher dataset 704. For instance, the teacher sample is a phenomic embedding generated by the teacher model 702 (e.g., a phenomic teacher machine learning model) and the batch correction technique 710 utilizes TVN.

Additionally, FIG. 7 shows the transcriptomic mapping system 102 using a third batch correction technique 714 to generate one or more third batch corrected data samples from the student dataset 708. In some embodiments, the transcriptomic mapping system 102 additionally uses a fourth batch correction technique 716 to generate one or more fourth batch corrected data samples from the student dataset 708. Furthermore, in some embodiments, the transcriptomic mapping system 102 also uses a fifth batch correction technique 718 to generate one or more fifth batch corrected data samples from the student dataset 708.

As described above in connection with FIG. 5, the transcriptomic mapping system 102 can select from a variety of batch correction techniques for this data augmentation process. For example, for each student sample in the student dataset 708, the transcriptomic mapping system 102 can randomly select the batch correction technique 714 from any of the above-described batch correction techniques. The transcriptomic mapping system 102 adds these batch corrected data samples to an augmented student biological dataset 724.

To further illustrate, the transcriptomic mapping system 102 generates a third batch corrected data sample by utilizing an identity model (e.g., the identity model 510), a feature centering model (e.g., the feature centering model 520), a center scaling model (e.g., the center scaling model 530), a typical variation normalization model (the TVN model 540), or a control sample centering model (e.g., the control sample centering model 550) to adjust a student sample from the student dataset 708. For instance, the student sample is a transcriptomic embedding generated by the student model 706 (e.g., a transcriptomic student machine learning model) and the batch correction technique 714 utilizes one of the above-described batch correction techniques.

Furthermore, in some embodiments, the transcriptomic mapping system 102 uses a mapping model 730 (e.g., the mapping model 320) to generate enhanced embeddings 732 from the data samples (e.g., transcriptomic embeddings) of the augmented student biological dataset 724. By applying batch correction techniques to augment the student dataset, in some implementations, the transcriptomic mapping system 102 enhances the robustness of the mapping model 730. To illustrate, the transcriptomic mapping system 102 provides varied training data to the mapping model 730 while maintaining the biological information in the data. Thus, the mapping model 730 learns to focus more on the biological information and less on non-biological information (e.g., batch effects and other variations).

Moreover, in some implementations, the transcriptomic mapping system 102 compares the enhanced embeddings 732 with the augmented teacher biological dataset to generate a measure of loss 740. Utilizing the measure of loss 740, the transcriptomic mapping system 102 adjusts parameters of the mapping model 730 to reduce the measure of loss 740 on a subsequent training iteration.

To further illustrate, in some embodiments, the transcriptomic mapping system 102 applies a fixed batch correction B to the teacher modality to ensure that only biologically relevant information is distilled through the training. This approach introduces distributional shifts while preserving the underlying biological information, encouraging the adapter f_{S} to learn representations with minimal batch-induced variability. Formally, the training objective (e.g., the measure of loss 740) is represented as

$$ℒ = - \frac{1}{N} {\sum }_{i = 1}^{N} [ log \frac{exp ( sim ( {f}_{S} ( A ( {z}_{S}^{( i )} ) ) , B ( {z}_{T}^{( i )} ) ) / τ )}{{\sum }_{j = 1}^{N} exp ( sim ( {f}_{S} ( A ( {z}_{S}^{( i )} ) ) , B ( {z}_{T}^{( j )} ) ) / τ )}$$

where A is randomly sampled from the set [symbol] for each data sample i.

During inference, in some embodiments, the transcriptomic mapping system 102 applies batch correction to the student representations to obtain

$${z}_{S , A}^{( i )}$$

before feeding them into the adapter f_{S} (e.g., the mapping model 730). Since the adapter f_{S} has been trained on these augmented representations, it effectively integrates the distilled knowledge from the teacher modality (e.g., phenomics) while filtering out batch-specific noise. Employing this strategy, the transcriptomic mapping system 102 can enhance the biological relevance of the student modality (e.g., transcriptomics) by focusing on shared biological information and improving robustness to experimental variability.

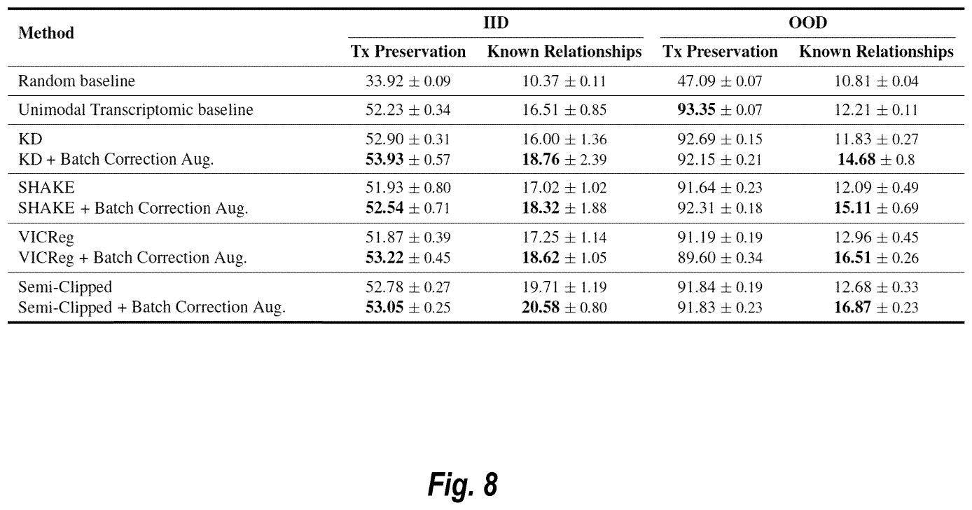

As discussed, the transcriptomic mapping system 102 improves performance over existing systems. FIG. 8 provides experimental results for the transcriptomic mapping system 102 in comparison with existing systems in accordance with one or more embodiments.

In particular, the results shown in FIG. 8 reflect experiments focused on microscopy imaging (phenomics) as a teacher modality and transcriptomics as a student modality. The training dataset consists of a curated set of paired samples from both modalities. The phenomics data includes 20,000 images of bulk cells (HUVEC cell line) acquired using cell painting and high-content screening, covering 1,700 unique chemical perturbations at three different concentrations per compound. The corresponding transcriptomics data includes bulk RNA sequencing from the same cell line (HUVEC), with 130K samples composed of the same 1,700 chemical perturbations and concentrations. Each sample from one modality can be paired with multiple samples from the other modality based on the same compound and concentration. During training, one corresponding pair is randomly selected for each sample from the student modality at each epoch.

Additionally, for the experiments represented in FIG. 8, pretrained encoders for both modalities are used to extract embeddings: z_{S} for the student (transcriptomics) and z_{T} for the teacher (phenomics). The student modality uses a three-layer MLP adapter f_{S} with ReLU activation functions, taking input embeddings in the transcriptomic feature space and outputting embeddings in the phenomic feature space. For these experiments, both the phenomics and transcriptomics encoders remain frozen during training, and only the MLP adapter for the student modality is trained. Additionally, the experiments include various batch correction techniques to improve robustness. For the teacher modality, the experiments use a TVN model (e.g., the TVN model 540 described above) as the batch correction function B. For the student modality, the experiments randomly apply one of the following during training: an identity model, a feature centering model, a center scaling model, or a TVN model (e.g., as described above in connection with FIG. 5). This augmentation helps the adapter learn representations resilient to distribution shift with invariant biological information. For the experiments represented in FIG. 8, the control samples are not used as paired data for cross-modal knowledge distillation, but only used for batch correction.

Evaluation for these experiments focuses on assessing the performance of the transcriptomics modality post knowledge distillation, with an objective to enhance the biological relevance of the transcriptomic representations without compromising their inherent interpretability. The primary tasks are: (1) unsupervised retrieval of known biological relationships to evaluate improvements in representation power and (2) linear interpretability using reconstruction to assess how well the distilled embeddings retain information for reconstructing raw gene expression counts. Success includes improving the retrieval task scores while maintaining performance on interpretability metrics at least similar to unimodal transcriptomic representations. This dual focus helps the student representations to not lose transcriptomic-specific information by simply copying the phenomics features.

Two separate evaluation settings are used to assess the model generalization. The first is an in-distribution (IID) dataset, consisting of transcriptomics data from the same cell line (HUVEC) and sequencing method as the training set. This dataset includes 300 distinct gene knock-out perturbations and contains 120K samples. It does not overlap with training data experiment batches. The second evaluation setting is an out-of-distribution (OOD) dataset based on different gene sequencing principles than those of the IID dataset. This dataset includes 443K samples from 31 distinct cell lines and features 5,157 genetic knockouts. This OOD dataset tests model robustness to new sequencing techniques and cell lines.

FIG. 8 shows comparisons of experimental results of the transcriptomic mapping system 102 knowledge distillation without batch correction as data augmentation (“Semi-Clipped”) with results from various existing systems. Additionally, FIG. 8 shows comparisons of the experimental results from the existing systems with augmented results of the existing systems augmented with the batch correction data augmentation techniques of the transcriptomic mapping system 102 described herein (“+Batch Correction Aug.” in FIG. 8). Furthermore, the last row of FIG. 8 shows the results of the transcriptomic mapping system 102 knowledge distillation with batch correction as data augmentation. For fair comparison, all approaches use the same pretrained unimodal encoders, with additional trainable adapters for the phenomics modality. The experiments are conducted with multiple seeds, and results are reported as averages with standard deviations. The reported scores are standardized as z-scores across methods and then averaged, with higher values indicating better performance.

As demonstrated in FIG. 8, the transcriptomic mapping system 102 provides improvements over existing systems both with its knowledge distillation techniques and its batch correction data augmentation techniques. Regarding the knowledge distillation techniques without data augmentation, the in-distribution experiments show that the transcriptomic mapping system 102 (“Semi-Clipped”) provides improved known relationship recall (“Known Relationships”) relative to a unimodal transcriptomic baseline and each of the existing systems (“KD”, “SHAKE”, and “VICReg”), while maintaining a comparable interpretability in transcriptomics (“Tx Preservation”). Thus, the transcriptomic mapping system 102 successfully distills biologically relevant information from phenomics to transcriptomics without loss of transcriptomic interpretability. In the out-of-distribution setting, the transcriptomic mapping system 102 performs competitively on relationship recall, surpassing label-dependent cross-modal methods and the unimodal baseline, while also closely matching the performance of other unsupervised multimodal methods. Additionally, the transcriptomic mapping system 102 performs competitively on transcriptomic preservation in the out-of-distribution setting. Overall, the transcriptomic mapping system 102 achieves a strong balance between generalization and interpretability preservation in both in-distribution and out-of-distribution settings.

As to batch correction as data augmentation, the transcriptomic mapping system 102 additionally provides improvements over existing systems. As shown in FIG. 8 on the rows labeled with “+Batch Correction Aug.”, the transcriptomic mapping system 102 improves performance both for the existing systems and for the knowledge distillation methods described herein, across both in-distribution and out-of-distribution settings. In particular, the known biological relationship recall scores improved across the board, while transcriptomic preservation scores either improved for the in-distribution scores and remained comparable for the out-of-distribution scores. For all methods, the improvements of relationship recall through batch correction augmentation were statistically significant compared to the scores without augmentations, with p-values below 0.05.

Notably, the last row of the table of FIG. 8 shows that the transcriptomic mapping system 102 provides enhanced performance when using both the knowledge distillation techniques and the batch correction as data augmentation techniques described herein. With both techniques applied, the transcriptomic mapping system 102 outperformed all other methods in known biological relationship recall for both in-distribution and out-of-distribution settings, while consistently preserving interpretability. Specifically, the transcriptomic mapping system 102 knowledge distillation approach with batch correction improved known biological relationship recall over the unimodal transcriptomics baseline by 24% in IID and 38% in OOD, thereby demonstrating enhanced accuracy for phenomics-to-transcriptomics distillation.

In addition, ablation studies were performed to identify the elements of the batch correction data augmentation methods that improve performance. The batch correction as data augmentation has two main parts: first, applying random batch correction to z_{S} student encoder embeddings during training, and second, applying TVN batch correction to z_{S} during inference before passing the embeddings to the adapter f_{S}. Following the experimental setup described above in connection with the table shown in FIG. 8, the known biological relationship recall was evaluated under several conditions: (1) the vanilla setup without data augmentations, (2) randomly applied batch correction augmentation during training, (3) TVN batch correction always applied only at inference, and (4) a complete bio-augmentation recipe with applying batch correction at both training and inference. Results are summarized in the following table.

| Configuration | KD | SHAKE | VICReg | Semi-Clipped |
|---|---|---|---|---|
| Vanilla | 16.00 | 17.02 | 17.25 | 19.71 |
| +Bio-Aug | 16.76 | 17.22 | 17.97 | 19.95 |
| +Inference on TVN | 17.36 | 17.66 | 18.19 | 19.34 |
| +Both | 18.76 | 18.32 | 18.62 | 20.58 |

The results show that each component provides performance improvements individually, with a slight exception for Semi-Clipped, where applying TVN correction at inference without augmentations led to a small decline. For all methods, using both components together consistently outperformed using either alone. This suggests that training with batch-corrected embeddings helps align the model's distribution with the TVN-corrected embeddings used in inference, mitigating possible distribution mismatch and preserving the benefits of TVN correction. Additionally, applying batch correction as a data augmentation, independently of using it for inference, increases diversity in training data without compromising biological relevance. This creates a range of embeddings with controlled biological information, which improves performance across all methods, unlike traditional augmentations, which may remove critical biological features.

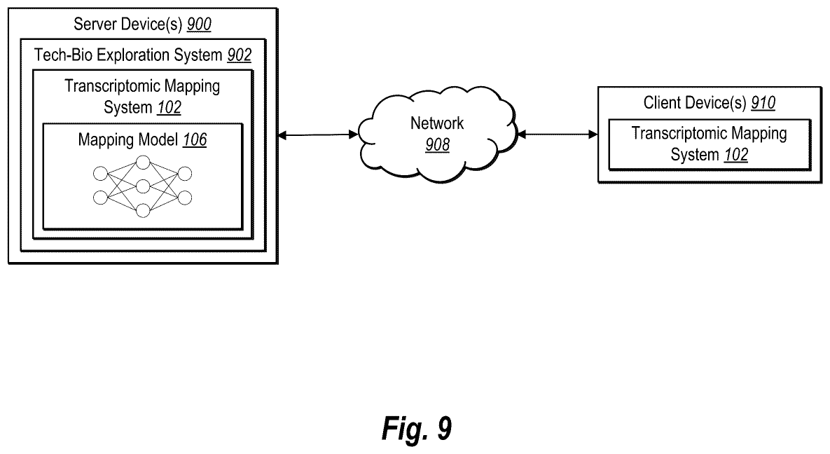

Additional detail regarding the computing environment in which the transcriptomic mapping system 102 operates will now be provided with reference to FIG. 9. In particular, FIG. 9 illustrates a schematic diagram of a system environment in which the transcriptomic mapping system 102 can operate in accordance with one or more embodiments.

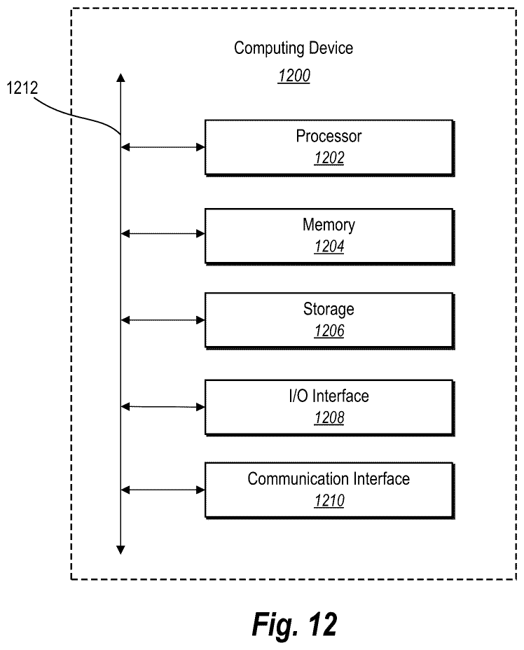

As shown in FIG. 9, the environment includes server device(s) 900 (which includes a tech-bio exploration system 902 and the transcriptomic mapping system 102), client device(s) 910, and a network 908. As further illustrated in FIG. 9, the various computing devices within the environment can communicate via the network 908. Although FIG. 9 illustrates the transcriptomic mapping system 102 being implemented by a particular component and/or device within the environment, the transcriptomic mapping system 102 can be implemented—in whole or in part—by other computing devices and/or components in the environment (e.g., additional client device(s)). Additional description regarding the illustrated computing devices is provided with respect to FIG. 12 below.

As shown in FIG. 9, the server device(s) 900 (e.g., one or more local servers operated by a particular entity) can include the tech-bio exploration system 902. In some embodiments, the tech-bio exploration system 902 can determine, store, generate, and/or provide for display tech-bio information including experiments from various sources, machine learning tech-bio predictions, machine learning embeddings for biological cell perturbations, and/or maps of biology, among others. For instance, the tech-bio exploration system 902 can analyze data signals corresponding to various treatments or interventions (e.g., compounds or biologics) and the corresponding relationships in genetics, proteomics, phenomics (e.g., cellular phenotypes), transcriptomics (e.g., transcription expression counts), and invivomics (e.g., expressions or results within a living animal). Moreover, the tech-bio exploration system 902 provides an environment for operating, executing, and/or managing complex drug discovery pipelines.

For instance, the tech-bio exploration system 902 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 902 can generate or determine a variety of predictions and inter-relationships for improving treatments/interventions.

To illustrate, the tech-bio exploration system 902 can train adapter models using cross modal knowledge distillation to improve biological representations by distilling previously unseen biological information into unimodal embeddings. For example, the tech-bio exploration system 902 can utilize machine learning to enhance biological information captured in data samples as part of the complex compound discovery process. For instance, the tech-bio exploration system 902 can identify a similarity between a first gene associated with disease treatment and a second gene previously unassociated with the disease based on a similarity in resulting phenotypes from gene knockout experiments. The tech-bio exploration system 902 can then identify new treatments based on the gene similarity (e.g., by targeting compounds that impact the second gene). Similarly, the tech-bio exploration system 902 can analyze signals from a variety of sources (e.g., protein interactions, in vivo experiments) to predict efficacious treatments based on various levels of biological data.

The tech-bio exploration system 902 can generate GUIs comprising dynamic user interface elements to convey tech-bio information and receive user input for intelligently exploring tech-bio information. Indeed, the tech-bio exploration system 902 can generate GUIs displaying biological information from a unimodal representation enhanced via an adapter model to convey additional biological information relating to another biological modality. Additionally, the tech-bio exploration system 902 can generate GUIs displaying augmented datasets generated from batch corrected data. Furthermore, the tech-bio exploration system 902 can also electronically communicate tech-bio information between various computing devices.

The tech-bio exploration system 902 can include a system that facilitates various models or algorithms for generating enhanced biological embeddings and discovering new treatment options over one or more networks. For example, the tech-bio exploration system 902 collects, manages, and transmits data across a variety of different entities, accounts, and devices. In some cases, the tech-bio exploration system 902 is a network system that facilitates access to (and analysis of) tech-bio information within a centralized operating system. Indeed, the tech-bio exploration system 902 can link data from different network-based research institutions to generate and analyze enhanced biological embeddings.

As shown in FIG. 9, the tech-bio exploration system 902 can include the transcriptomic mapping system 102 that generates, stores, manages, and/or transmits data pertaining to biological embeddings. For example, in the context of the above description for the tech-bio exploration system 902, in some embodiments, the tech-bio exploration system 902 further utilizes the transcriptomic mapping system 102 to enhance the coordination between various groups involved in the drug discovery process. For instance, the transcriptomic mapping system 102 works in tandem with the tech-bio exploration system 902 to generate enhanced biological embeddings, generate augmented biological datasets, transmit the enhanced biological embeddings and/or the augmented biological datasets to one or more devices, and initiate one or more downstream model predictions or processes.

As also illustrated in FIG. 9, the environment includes the client device(s) 910. As mentioned above, the client device(s) 910 can be involved in the process of drug discovery. Thus, for example, the client device(s) 910 can coordinate/manage a first stage of generating enhanced biological embeddings. Moreover, the client device(s) 910 can coordinate/manage a second stage such as generating a biological activity prediction based on one or more enhanced biological embeddings. Further, the client device(s) 910 can coordinate and/or manage a third stage of utilizing the biological activity prediction to generate one or more additional predictions or initiate one or more programs (e.g., industrial program generation (IPG) or industrialized compound generation (ICG)).

To illustrate, the client device(s) 910 can include computing devices that implement or manage a compound program generation stage of a compound discovery process. Similarly, the client device(s) 910 can include computing devices that implement or manage a compound lead generation stage and the client device(s) 910 can include computing devices that implement or manage a compound/dose selection stage. For example, the transcriptomic mapping system 102 can receive one or more requests to utilize the mapping model 106 to generate one or more enhanced biological embeddings. For instance, the transcriptomic mapping system 102 can receive additional requests from the client device(s) 910 that include generating the biological activity predictions.

In some embodiments, the environment also includes additional device(s). For example, the transcriptomic mapping system 102 can utilize the additional device(s) to further operate and manage the completion of complex drug discovery pipelines. For instance, the additional device(s) include experimental device(s) and analytical device(s). Further, in some instances, the additional device(s) also include the computing devices discussed below in connection with FIG. 12.

Furthermore, in one or more implementations, the client device(s) 910 include a client application. The client application can include instructions that (upon execution) cause the client device(s) 910 to perform various actions. For example, a user of a user account can interact with the client application on the client device(s) 910 to execute experiments or other multi-faceted processes, to further access tech-bio information, and/or initiate a request for a biological activity prediction. For instance, in some embodiments the transcriptomic mapping system 102 receives a request to generate an enhanced biological embedding, and in response generates the enhanced biological embedding and returns the enhanced biological embedding to the client device(s) 910. In some instances, the transmittal of the enhanced biological embedding to the client device(s) 910 causes the client device(s) 910 to execute an action (e.g., generate a downstream model prediction or other task).

Additionally, the environment can include dedicated machine learning device(s). For example, the dedicated machine learning device(s) can include computing devices or virtual machines dedicated to training or implementing large-scale machine learning models. For example, the dedicated machine learning device(s) can generate machine learning predictions and/or embeddings based on digital biological data (e.g., digital images of phenotypes resulting from different perturbations or compound-protein interactions from compound features, transcriptomic profiles of transcription expression counts for different perturbations or compound-protein interactions, etc.). Thus, the transcriptomic mapping system 102 can interact with the dedicated machine learning device(s) to generate enhanced biological embeddings.

The environment can also include experimental device(s). For example, the tech-bio exploration system 902 can interact with experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of in vivo experimentation. The tech-bio exploration system 902 can also interact with a variety of other experimental device(s) such as devices for determining, generating, or extracting gene sequences or protein information. For example, the experimental device(s) may include computing devices linked to biosensors, electrophysiological platforms, x-ray crystallography machines, liquid chromatography mass spectrometry systems, nuclear magnetic resonance spectrometers, and/or mass spectrometers. In some implementations, the transcriptomic mapping system 102 generates enhanced biological embeddings and further determines to employ or utilize one or more experimental devices (e.g., to initiate one or more experiments based on the similarity predictions).

As further shown in FIG. 9, the environment includes the network 908. As mentioned above, the network 908 can enable communication between components of the environment. In one or more embodiments, the network 908 may include a suitable network and may communicate using a various number of communication platforms and technologies suitable for transmitting data and/or communication signals, examples of which are described with reference to FIG. 12. Furthermore, although FIG. 9 illustrates computing devices communicating via the network 908, the various components of the environment can communicate and/or interact via other methods (e.g., communicate directly).

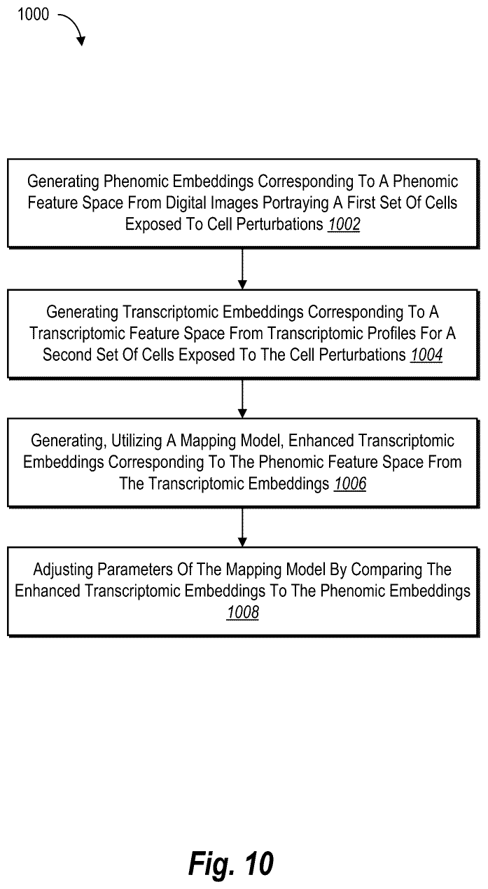

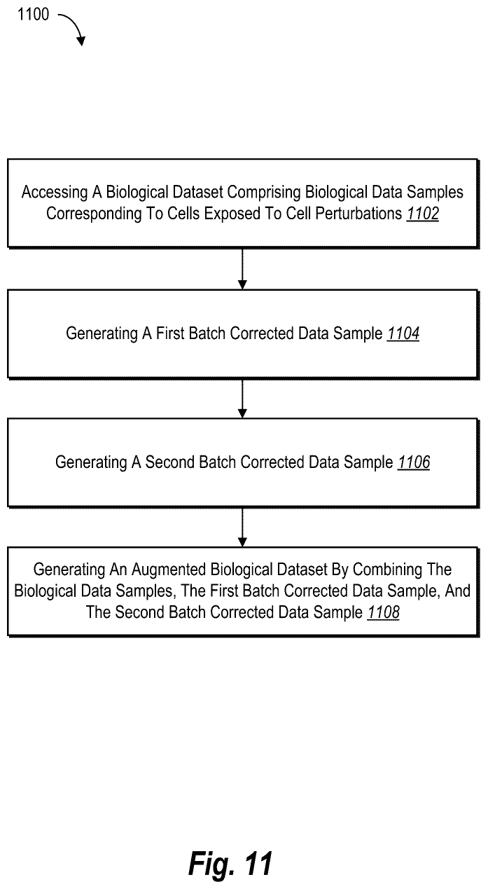

FIGS. 1-9, the corresponding text, and the examples provide a number of different methods, systems, devices, and non-transitory computer-readable media of the transcriptomic mapping system 102. In addition to the foregoing, one or more embodiments are described in terms of flowcharts comprising acts for accomplishing a particular result, as shown in FIGS. 10 and 11. In some implementations, the processes of the transcriptomic mapping system 102 are performed with more or fewer acts. Furthermore, in various implementations, the acts are performed in differing orders. Additionally, in some implementations, the acts described herein are repeated or performed in parallel with one another or in parallel with different instances of the same or similar acts.

As mentioned, FIG. 10 illustrates a flowchart of a series of acts 1000 for generating an enhanced biological embedding and training a mapping model based on the enhanced biological embedding in accordance with one or more implementations. Additionally, FIG. 11 illustrates a flowchart of a series of acts 1100 for generating an augmented biological dataset in accordance with one or more implementations. While FIGS. 10 and 11 illustrate acts according to various implementations, alternative implementations omit, add to, reorder, and/or modify any of the acts shown in FIGS. 10 and 11. In one or more implementations, the acts of FIGS. 10 and 11 are performed as part of a method (e.g., a computer-implemented method). Alternatively, in one or more implementations, a non-transitory computer-readable storage medium comprises instructions that, when executed by one or more processors, cause a computing device to perform the acts of FIGS. 10 and 11. In some implementations, a system performs the acts of FIGS. 10 and 11.

As shown in FIG. 10, the series of acts 1000 includes an act 1002 of generating phenomic embeddings corresponding to a phenomic feature space from digital images portraying a first set of cells exposed to cell perturbations, an act 1004 of generating transcriptomic embeddings corresponding to a transcriptomic feature space from transcriptomic profiles for a second set of cells exposed to the cell perturbations, an act 1006 of generating, utilizing a mapping model, enhanced transcriptomic embeddings corresponding to the phenomic feature space from the transcriptomic embeddings, and an act 1008 of adjusting parameters of the mapping model by comparing the enhanced transcriptomic embeddings to the phenomic embeddings.

In particular, in some implementations, the act 1002 includes generating, utilizing a phenomic teacher machine learning model, phenomic embeddings corresponding to a phenomic feature space from digital images portraying a first set of cells exposed to cell perturbations, the act 1004 includes generating, utilizing a transcriptomic student machine learning model, transcriptomic embeddings corresponding to a transcriptomic feature space from transcriptomic profiles for a second set of cells exposed to the cell perturbations, the act 1006 includes generating, utilizing a mapping model, enhanced transcriptomic embeddings corresponding to the phenomic feature space from the transcriptomic embeddings corresponding to the transcriptomic feature space, and the act 1008 includes adjusting parameters of the mapping model by comparing the enhanced transcriptomic embeddings to the phenomic embeddings.

For example, in some implementations, the series of acts 1000 includes accessing a transcriptomic embedding for a cell exposed to a cell perturbation. Additionally, in some implementations, the series of acts 1000 includes generating, from the transcriptomic embedding utilizing the mapping model, an enhanced transcriptomic embedding corresponding to the phenomic feature space. Moreover, in some implementations, the series of acts 1000 includes generating the transcriptomic profiles for the second set of cells by generating a data structure of transcription expression counts for the cell perturbations.

Furthermore, in some implementations, the series of acts 1000 includes generating the enhanced transcriptomic embeddings by generating a feature vector that retains transcriptomic interpretability for generating a transcriptomic profile from the feature vector. In addition, in some implementations, the series of acts 1000 includes adjusting the parameters of the mapping model by: fixing the phenomic embeddings generated by the phenomic teacher machine learning model; and adjusting the parameters of the mapping model to learn to map transcriptomic embeddings to the phenomic feature space.

Moreover, in some implementations, the series of acts 1000 includes adjusting the parameters of the mapping model by: determining a measure of loss by comparing a first enhanced transcriptomic embedding for a first cell perturbation to a first phenomic embedding for the first cell perturbation; and adjusting the parameters of the mapping model to improve the measure of loss. Furthermore, in some implementations, the series of acts 1000 includes adjusting the parameters of the mapping model further by: determining the measure of loss by comparing a second enhanced transcriptomic embedding for a second cell perturbation to a second phenomic embedding for the second cell perturbation; and adjusting the parameters of the mapping model to improve the measure of loss. Additionally, in some implementations, the series of acts 1000 includes comparing the enhanced transcriptomic embeddings to the phenomic embeddings by determining cosine similarities between the enhanced transcriptomic embeddings and the phenomic embeddings.

As shown in FIG. 11, the series of acts 1100 includes an act 1102 of accessing a biological dataset comprising biological data samples corresponding to cells exposed to cell perturbations, an act 1104 of generating a first batch corrected data sample, an act 1106 of generating a second batch corrected data sample, and an act 1108 of generating an augmented biological dataset by combining the biological data samples, the first batch corrected data sample, and the second batch corrected data sample.

In particular, in some implementations, the act 1102 includes accessing a biological dataset comprising biological data samples corresponding to cells exposed to cell perturbations, the act 1104 includes generating, utilizing a first batch correction technique for a first sample of the biological data samples, a first batch corrected data sample, the act 1106 includes generating, utilizing a second batch correction technique for a second sample of the biological data samples, a second batch corrected data sample, and the act 1108 includes generating, from the biological dataset, an augmented biological dataset for training a machine learning model by combining the biological data samples, the first batch corrected data sample, and the second batch corrected data sample.

For example, in some implementations, the series of acts 1100 includes generating, utilizing a third batch correction technique for a third sample of the biological data samples, a third batch corrected data sample. Additionally, in some implementations, the series of acts 1100 includes generating the augmented biological dataset by combining the biological data samples, the first batch corrected data sample, the second batch corrected data sample, and the third batch corrected data sample.

Moreover, in some implementations, the series of acts 1100 includes generating the biological data samples by generating, utilizing an embedding model, embeddings for the cells exposed to the cell perturbations. In addition, in some implementations, the series of acts 1100 includes generating the first batch corrected data sample for the first sample by generating, utilizing the first batch correction technique, a first batch corrected embedding from the embeddings. Furthermore, in some implementations, the series of acts 1100 includes generating, utilizing the machine learning model, a biological prediction for a cell perturbation from the first batch corrected embedding. In addition, in some implementations, the series of acts 1100 includes adjusting parameters of the machine learning model by comparing the biological prediction to a ground truth. Additionally, in some implementations, the series of acts 1100 includes adjusting the parameters of the machine learning model by adjusting parameters of a mapping model for converting transcriptomic embeddings to a phenomic embedding space.

Moreover, in some implementations, the series of acts 1100 includes utilizing the first batch correction technique by utilizing an identity model, a feature centering model, or a center scaling model. Additionally, in some implementations, the series of acts 1100 includes utilizing the second batch correction technique by utilizing a typical variation normalization model or a control sample centering model. Furthermore, in some implementations, the series of acts 1100 includes generating the first batch corrected data sample by utilizing a typical variation normalization model to adjust a teacher sample generated by a phenomic teacher machine learning model. In addition, in some implementations, the series of acts 1100 includes generating the second batch corrected data sample by utilizing an identity model, a feature centering model, a center scaling model, the typical variation normalization model, or a control sample centering model to adjust a student sample generated by a transcriptomic student machine learning model.

Embodiments of the present disclosure may comprise or utilize a special purpose or general purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions from a non-transitory computer-readable medium (e.g., memory) and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or generators and/or other electronic devices. When information is transferred, or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface generator (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general purpose computer, special purpose computer, or special purpose processing device to perform a certain function or group of functions. In some embodiments, computer-executable instructions are executed by a general purpose computer to turn the general purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program generators may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), a web service, Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 12 illustrates a block diagram of an example computing device 1200 that may be configured to perform one or more of the processes described above. One will appreciate that one or more computing devices, such as the computing device 1200, may represent the computing devices described above (e.g., the server device(s) 900 or the client device(s) 910). In one or more embodiments, the computing device 1200 may be a mobile device (e.g., a mobile telephone, a smartphone, a PDA, a tablet, a laptop, a camera, a tracker, a watch, a wearable device, etc.). In some embodiments, the computing device 1200 may be a non-mobile device (e.g., a desktop computer or another type of client device). Further, the computing device 1200 may be a server device that includes cloud-based processing and storage capabilities.

As shown in FIG. 12, the computing device 1200 can include one or more processor(s) 1202, memory 1204, a storage device 1206, input/output interfaces 1208 (or “I/O interfaces 1208”), and a communication interface 1210, which may be communicatively coupled by way of a communication infrastructure (e.g., bus 1212). While the computing device 1200 is shown in FIG. 12, the components illustrated in FIG. 12 are not intended to be limiting. Additional or alternative components may be used in other embodiments. Furthermore, in certain embodiments, the computing device 1200 includes fewer components than those shown in FIG. 12. Components of the computing device 1200 shown in FIG. 12 will now be described in additional detail.

In particular embodiments, the processor(s) 1202 includes hardware for executing instructions, such as those making up a computer program. As an example, and not by way of limitation, to execute instructions, the processor(s) 1202 may retrieve (or fetch) the instructions from an internal register, an internal cache, memory 1204, or a storage device 1206 and decode and execute them.

The computing device 1200 includes the memory 1204, which is coupled to the processor(s) 1202. The memory 1204 may be used for storing data, metadata, and programs for execution by the processor(s). The memory 1204 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1204 may be internal or distributed memory.

The computing device 1200 includes the storage device 1206 for storing data or instructions. As an example, and not by way of limitation, the storage device 1206 can include a non-transitory storage medium described above. The storage device 1206 may include a hard disk drive (“HDD”), flash memory, a Universal Serial Bus (“USB”) drive or a combination these or other storage devices.

As shown, the computing device 1200 includes one or more I/O interfaces 1208, which are provided to allow a user to provide input to (such as user strokes), receive output from, and otherwise transfer data to and from the computing device 1200. These I/O interfaces 1208 may include a mouse, keypad or a keyboard, a touch screen, camera, optical scanner, network interface, modem, other known I/O devices or a combination of such I/O interfaces 1208. The touch screen may be activated with a stylus or a finger.

The I/O interfaces 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1208 are configured to provide graphical data to a display for presentation to a user. The graphical data may be representative of one or more graphical user interfaces and/or any other graphical content as may serve a particular implementation.

The computing device 1200 can further include a communication interface 1210. The communication interface 1210 can include hardware, software, or both. The communication interface 1210 provides one or more interfaces for communication (such as, for example, packet-based communication) between the computing device and one or more other computing devices or one or more networks. As an example, and not by way of limitation, communication interface 1210 may include a network interface controller (“NIC”) or network adapter for communicating with an Ethernet or other wire-based network or a wireless NIC (“WNIC”) or wireless adapter for communicating with a wireless network, such as a WI-FI. The computing device 1200 can further include the bus 1212. The bus 1212 can include hardware, software, or both that connects components of computing device 1200 to each other.

The components of the transcriptomic mapping system 102 include software, hardware, or both. For example, the components include one or more instructions stored on a computer-readable storage medium and executable by processors of one or more computing devices, such as a client device or server device. When executed by the one or more processors, in some implementations, the computer-executable instructions of the transcriptomic mapping system 102 cause the computing device(s) to perform the methods described herein. Alternatively, in one or more implementations, the components include hardware, such as a special purpose processing device to perform a certain function or group of functions. Alternatively, in some implementations, the components of the transcriptomic mapping system 102 include a combination of computer-executable instructions and hardware.

Furthermore, the components of the transcriptomic mapping system 102 are, for example, implemented as one or more operating systems, as one or more stand-alone applications, as one or more modules of an application, as one or more plug-ins, as one or more library functions, as one or more functions callable by other applications, and/or as a cloud-computing model. Thus, in some implementations, the components are implemented as a stand-alone application, such as a desktop or mobile application. Furthermore, in various implementations, the components are implemented as one or more web-based applications hosted on a remote server. In some implementations, the components are implemented in a suite of mobile device applications or “apps.”

The use in the foregoing description and in the appended claims of the terms “first,” “second,” “third,” etc., is not necessarily to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget, and not necessarily to connote that the second widget has two sides.

In the foregoing description, the invention has been described with reference to specific exemplary embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
accessing a biological dataset comprising biological data samples corresponding to cells exposed to cell perturbations;
generating, utilizing a first batch correction technique for a first sample of the biological data samples, a first batch corrected data sample;
generating, utilizing a second batch correction technique for a second sample of the biological data samples, a second batch corrected data sample; and
generating, from the biological dataset, an augmented biological dataset for training a machine learning model by combining the biological data samples, the first batch corrected data sample, and the second batch corrected data sample.

2. The computer-implemented method of claim 1, further comprising:
generating, utilizing a third batch correction technique for a third sample of the biological data samples, a third batch corrected data sample; and
generating the augmented biological dataset by combining the biological data samples, the first batch corrected data sample, the second batch corrected data sample, and the third batch corrected data sample.

3. The computer-implemented method of claim 1, further comprising:
generating the biological data samples by generating, utilizing an embedding model, embeddings for the cells exposed to the cell perturbations; and
generating the first batch corrected data sample for the first sample by generating, utilizing the first batch correction technique, a first batch corrected embedding from the embeddings.

4. The computer-implemented method of claim 3, further comprising:
generating, utilizing the machine learning model, a biological prediction for a cell perturbation from the first batch corrected embedding; and
adjusting parameters of the machine learning model by comparing the biological prediction to a ground truth.

5. The computer-implemented method of claim 4, wherein adjusting the parameters of the machine learning model comprises adjusting parameters of a mapping model for converting transcriptomic embeddings to a phenomic embedding space.

6. The computer-implemented method of claim 1, wherein utilizing the first batch correction technique comprises utilizing an identity model, a feature centering model, or a center scaling model, and
wherein utilizing the second batch correction technique comprises utilizing a typical variation normalization model or a control sample centering model.

7. The computer-implemented method of claim 1, wherein:
generating the first batch corrected data sample comprises utilizing a typical variation normalization model to adjust a teacher sample generated by a phenomic teacher machine learning model; and
generating the second batch corrected data sample comprises utilizing an identity model, a feature centering model, a center scaling model, the typical variation normalization model, or a control sample centering model to adjust a student sample generated by a transcriptomic student machine learning model.

8. A system comprising:
at least one processor; and
at least one non-transitory computer-readable storage medium storing instructions that, when executed by the at least one processor, cause the system to:
access a biological dataset comprising biological data samples corresponding to cells exposed to cell perturbations;
generate, utilizing a first batch correction technique for a first sample of the biological data samples, a first batch corrected data sample;
generate, utilizing a second batch correction technique for a second sample of the biological data samples, a second batch corrected data sample; and
generate, from the biological dataset, an augmented biological dataset for training a machine learning model by combining the biological data samples, the first batch corrected data sample, and the second batch corrected data sample.

9. The system of claim 8, wherein the at least one non-transitory computer-readable storage medium stores additional instructions that, when executed by the at least one processor, cause the system to:
generate, utilizing a third batch correction technique for a third sample of the biological data samples, a third batch corrected data sample; and
generate the augmented biological dataset by combining the biological data samples, the first batch corrected data sample, the second batch corrected data sample, and the third batch corrected data sample.

10. The system of claim 8, wherein the at least one non-transitory computer-readable storage medium stores additional instructions that, when executed by the at least one processor, cause the system to:
generate the biological data samples by generating, utilizing an embedding model, embeddings for the cells exposed to the cell perturbations; and
generate the first batch corrected data sample for the first sample by generating, utilizing the first batch correction technique, a first batch corrected embedding from the embeddings.

11. The system of claim 10, wherein the at least one non-transitory computer-readable storage medium stores additional instructions that, when executed by the at least one processor, cause the system to:
generate, utilizing the machine learning model, a biological prediction for a cell perturbation from the first batch corrected embedding; and
adjust parameters of the machine learning model by comparing the biological prediction to a ground truth.

12. The system of claim 11, wherein the at least one non-transitory computer-readable storage medium stores additional instructions that, when executed by the at least one processor, cause the system to adjust the parameters of the machine learning model by adjusting parameters of a mapping model for converting transcriptomic embeddings to a phenomic embedding space.

13. The system of claim 8, wherein the at least one non-transitory computer-readable storage medium stores additional instructions that, when executed by the at least one processor, cause the system to:
utilize the first batch correction technique by utilizing an identity model, a feature centering model, or a center scaling model; and
utilize the second batch correction technique by utilizing a typical variation normalization model or a control sample centering model.

14. The system of claim 8, wherein the at least one non-transitory computer-readable storage medium stores additional instructions that, when executed by the at least one processor, cause the system to:
generate the first batch corrected data sample by utilizing a typical variation normalization model to adjust a teacher sample generated by a phenomic teacher machine learning model; and
generate the second batch corrected data sample by utilizing an identity model, a feature centering model, a center scaling model, the typical variation normalization model, or a control sample centering model to adjust a student sample generated by a transcriptomic student machine learning model.

15. A non-transitory computer-readable medium storing instructions that, when executed by at least one processor, cause a computing device to:
access a biological dataset comprising biological data samples corresponding to cells exposed to cell perturbations;
generate, utilizing a first batch correction technique for a first sample of the biological data samples, a first batch corrected data sample;
generate, utilizing a second batch correction technique for a second sample of the biological data samples, a second batch corrected data sample; and
generate, from the biological dataset, an augmented biological dataset for training a machine learning model by combining the biological data samples, the first batch corrected data sample, and the second batch corrected data sample.

16. The non-transitory computer-readable medium of claim 15, further storing additional instructions that, when executed by the at least one processor, cause the computing device to:
generate the biological data samples by generating, utilizing an embedding model, embeddings for the cells exposed to the cell perturbations; and
generate the first batch corrected data sample for the first sample by generating, utilizing the first batch correction technique, a first batch corrected embedding from the embeddings.

17. The non-transitory computer-readable medium of claim 16, further storing additional instructions that, when executed by the at least one processor, cause the computing device to:
generate, utilizing the machine learning model, a biological prediction for a cell perturbation from the first batch corrected embedding; and
adjust parameters of the machine learning model by comparing the biological prediction to a ground truth.

18. The non-transitory computer-readable medium of claim 17, further storing additional instructions that, when executed by the at least one processor, cause the computing device to adjust the parameters of the machine learning model by adjusting parameters of a mapping model for converting transcriptomic embeddings to a phenomic embedding space.

19. The non-transitory computer-readable medium of claim 15, further storing additional instructions that, when executed by the at least one processor, cause the computing device to:
utilize the first batch correction technique by utilizing an identity model, a feature centering model, or a center scaling model; and
utilize the second batch correction technique by utilizing a typical variation normalization model or a control sample centering model.

20. The non-transitory computer-readable medium of claim 15, further storing additional instructions that, when executed by the at least one processor, cause the computing device to:
generate the first batch corrected data sample by utilizing a typical variation normalization model to adjust a teacher sample generated by a phenomic teacher machine learning model; and
generate the second batch corrected data sample by utilizing an identity model, a feature centering model, a center scaling model, the typical variation normalization model, or a control sample centering model to adjust a student sample generated by a transcriptomic student machine learning model.
